(19) United States
(12) Patent Application Publication
(10) Pub. No.: US 2007/0100446 A1
(43) Pub. Date: May 3, 2007

(54) INTRAOCULAR LENS

(76) Inventor: Donald Horvatich, Alta Loma, CA (US)

Correspondence Address: KING & SCHICKLI, PLLC, 247 NORTH BROADWAY, LEXINGTON, KY, 40507, US

(21) Appl. No.: 11/553,004

(22) Filed: Oct. 26, 2006

Related U.S. Application Data
(60) Provisional application No. 60/730,733, filed on Oct. 27, 2005.

(51) Int. Cl. A61F 2/16

(52) U.S. Cl. 623/6.44; 623/6.51

(57) ABSTRACT

A lens for an eye including an optic element and a plurality of supports connected to the optic element, wherein the lens may be implanted in the eye. The plurality of supports may be symmetrical and each may include an aperture. A related method is also provided.

This application claims the benefit of U.S. Provisional Patent Application No. 60/730,733 filed Oct. 27, 2005, the disclosure of which is herein incorporated by reference.

### TECHNICAL FIELD

The present invention relates generally to the ophthalmological arts and, more specifically, to an intraocular lens having lateral supports.

### BACKGROUND OF THE INVENTION

The human eye includes a transparent lens positioned behind the iris to focus light rays onto the retina. As humans age, this lens loses clarity and becomes clouded. This lens condition, sometimes referred to as a cataract, results in decreased vision or even blindness in the affected eye.

The predominant method for treating a cataract is to remove the cloudy lens from the eye. However, once the natural lens of the eye becomes removed, an artificial lens is necessary to return vision to the eye. A well known method for returning vision is to implant a permanent prescription lens, sometimes referred to as an intraocular lens (“IOL”).

Generally, the eye surgeon will make an incision in the cornea of the eye and the capsular bag that holds the natural lens. After removal of the natural lens, the surgeon may insert the IOL into the capsular bag. Several methods are known for cutting and inserting the IOL. One popular method is to make a small incision (˜2.5 mm in length) in the eye, fold or roll the IOL, and insert it through the small incision into the capsular bag. Once inserted, the IOL naturally unfurls itself into a desired position within the capsular bag. Given the small size of the incision, the natural internal pressure of the eye holds the incision closed, thereby possibly alleviating the need for stitches to close the incision.

Many different configurations of IOLs have been developed. These previous designs often include two arcuate cantilevered support extending from the optic element of the lens. Once inserted, this lens requires large scale bending of the support to maintain the optic element in the desired position for focal correction. Unfortunately, these configurations do not provide the desired to support for the capsular bag, This often results in an increased amount of capsular prolapse.

Accordingly, the ophthalmological arts have need for a IOL that includes increased supports for supporting the optic element, as well as the capsular bag.

### SUMMARY OF THE INVENTION

In accordance with one aspect of the invention, a lens for an eye is disclosed. The lens comprises an optic element and a plurality of symmetrical supports connected to the optic element, wherein the lens may be implanted in the eye.

In one embodiment, the plurality of symmetrical supports are flexible and the supports include two supports positioned on opposite sides of the optic element. The optic element may be formed from first and second curved surfaces on opposite sides of a body. The plurality of symmetrical supports may include a first leg and a second leg extending away from a body. The first and second legs may diverge from a center line of the body.

In accordance with another aspect of the invention, a lens for an eye is disclosed. The lens comprises a body having a curved optic element and a first and second support connected to the optic element. Each support has a plurality of leg portions and the first support includes a first aperture, whereby the lens may be implanted in the eye. The optic element may comprise first and second curved surfaces formed on opposite sides of the body. The optic element may be positioned substantially in the center of the body. The first and second supports may be symmetrical. The second support may include an aperture and the first and second supports may bend in a direction away from a plane defined by the body. Each of the leg portions may terminate in a foot portion. A first member may connect the foot portions of the first support and a second member may connect the foot portions of the second support. The first and second members may be curved and the legs of the first support and first member may define the first aperture. The legs of the second support and second curved member may define a second aperture. The apertures may be substantially triangular.

In accordance with another aspect of the invention, a lens for an eye is disclosed. The lens comprises a body having a curved optic element and first and second symmetrical supports connected to the body. Each support may include an aperture and each support may terminate in a curved member, wherein the lens may be implanted in the eye. The body may have both horizontal and vertical symmetry. Each support may include at least one foot portion that extends beyond a radius of the optic element. Each support may include an aperture.

In accordance with another aspect of the invention, a lens for an eye is disclosed. The lens comprises a body having first and second curved optic elements. The optic elements are positioned on opposite sides of the body. First and second symmetrical supports are connected to the body, each support including a plurality of legs, each of the legs curving away from a center line of the body. The lens includes a foot portion positioned at the end of each leg. A first member resides between and connects each of the foot portions of the first support and a second member resides between and connects each of the foot portions of the second support, wherein each of the first and second symmetrical supports includes an aperture and the lens may be implanted in the eye. Each the foot portions may extend beyond a radius of the optic elements. The first and second symmetrical supports may be flexible, the first and second members may be curved, and the apertures may substantially triangular.

In accordance with another aspect of the invention, a method of implanting a lens inside an eye is disclosed. The method comprises making an incision in a cornea or sclera of the eye and inserting the lens into a capsular bag of the eye, wherein the lens includes an optic element and a plurality of symmetrical supports carried by the lens. The inserting step may comprise rolling the lens and placing it into an injector. The inserting step may comprise folding the lens and placing it into an injector.

The following drawings pertain to one possible embodiment of this invention, and are merely designed to illustrate one of the modes best suited to carry out the invention. As it will be realized, the invention is capable of other different embodiments, and its several details are capable of modification in various, obvious aspects all without departing from the invention. Accordingly, the drawings and descriptions will be regarded as illustrative in nature and not as restrictive.

### BRIEF DESCRIPTION OF THE DRAWINGS

The accompanying drawings incorporated in and forming a part of the specification, illustrate several aspects of the present invention, and together with the description serve to explain the principles of the invention.

In the drawings:

FIG. 1a is a perspective view of an intraocular lens forming one possible embodiment of the present invention;

FIG. 1b is a front view of the lens of FIG. 1a;

FIG. 1c is a side view of the lens of FIG. 1a;

FIG. 2 is a diagrammatic view of an eye and one possible embodiment of installation of the lens of FIG. 1a; and

FIG. 3 is a diagrammatic view of an eye with the lens of FIG. 1a installed.

### DETAILED DESCRIPTION OF THE INVENTION

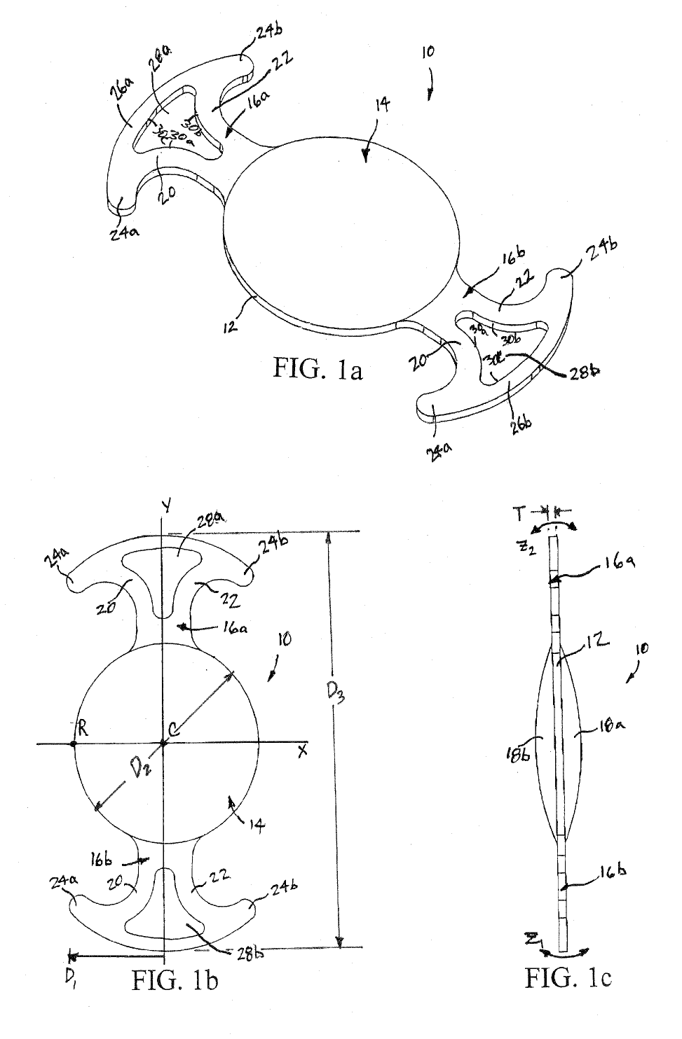

FIGS. 1a-3 illustrate one embodiment of the intraocular lens (“IOL”) 10 of the present invention. The IOL 10 becomes installed in a capsular bag 34 of the eye E to restore vision to an aphakic human eye. With specific reference to FIGS. 1a-1c, the IOL 10 includes a main body 12 having an optic element 14 and a pair of supports 16a, 16b.

As shown in FIG. 1c, the optic element 14 includes first and second curved surfaces 18a, 18b positioned on opposite sides of the main body 12. The first and second curved surfaces 18a, 18b of optic element 14 form the lens that serves as a replacement for the cataract lens removed from the eye. Similar to previously known IOLs, the amount of curvature may be varied to meet the needs of the recipient and provide optimized vision. Preferably the curved surfaces 18a, 18b lie intermediate of the supports 16a, 16b. This enables the optic element 14 to become positioned in a plane appropriate for focal correction of the eye.

To assist in properly positioning the optic element 14, the supports 16a, 16b are preferably positioned symmetrically on the main body 12 in an opposed fashion. Each support 16a, 16b includes a first leg 20 and a second leg 22 extending away from the main body 12 in an opposed fashion. As shown in FIG. 1b, the first and second legs 20, 22 divergently curve away from a center line Y. Additionally, each leg 20, 22 terminates in foot portions 24a, 24b, respectively. Preferably, the foot portions 24a, 24b extend a distance D₁ away from the center line Y. The distance D₁ is equal to or slightly larger than a radius between the center point C of the optic element 14 and the point R. This distance D₁ of the foot portions 24a, 24b helps to disperse the force of the supports 16a, 16b to a greater area of the capsular bag 34, which assists in supporting optic element 14 and the capsular bag 34.

One will appreciate that the present configuration provides the IOL 10 with symmetry about the horizontal axis X and the vertical axis (represented by the center line Y). The symmetry of the supports 16a, 16b provides an even amount of force on the optic element 14 when installed. This helps maintain the IOL 10 and the optic element 14 in the desired plane appropriate for focal correction of the eye. Additionally, this helps support the capsular bag 34, thereby minimizing prolapse, as discussed below.

The supports 16a, 16b also include curved members 26a, 26b residing between and connecting the foot portions 24a, 24b. The curved members 26a, 26b assist in providing some structural rigidity to the supports 16a, 16b. The curved members 26a, 26b also engage and provide support for the capsular bag 34, when installed. As shown, the legs 20, 22 and curved members 26a, 26b define apertures 28a, 28b. In one embodiment, the apertures 28a, 28b have a somewhat triangular appearance with the legs 20, 22 and curved member 26 providing the apertures 28a, 28b with curved surfaces 30a, 30b, 30c. The apertures 28a, 28b enable the supports 16a, 16b to slightly bend in the directions Z₁, Z₂ (FIG. 1c). This bending provides outward force for assisting in support of the optic element 14, as well as the capsular bag 34, when installed.

The IOL 10 may have any dimensions desired and be formed of any material that enables insertion in the capsular bag 34 and penetration of light through the optic element 14. In one embodiment, the optic element of the IOL 10 has a diameter D₂ of approximately 6.0 mm and an overall diameter D₃ of 12.5 mm. The IOL 10 may also have a thickness T of 0.25 mm (FIG. 1c). In this embodiment, the IOL 10 is formed from silicon or any hydrophilic or hydrophobic material, such as an acrylate. Additionally, the IOL 10 may be formed as a single piece of material or multiple pieces joined together by methods known in the art.

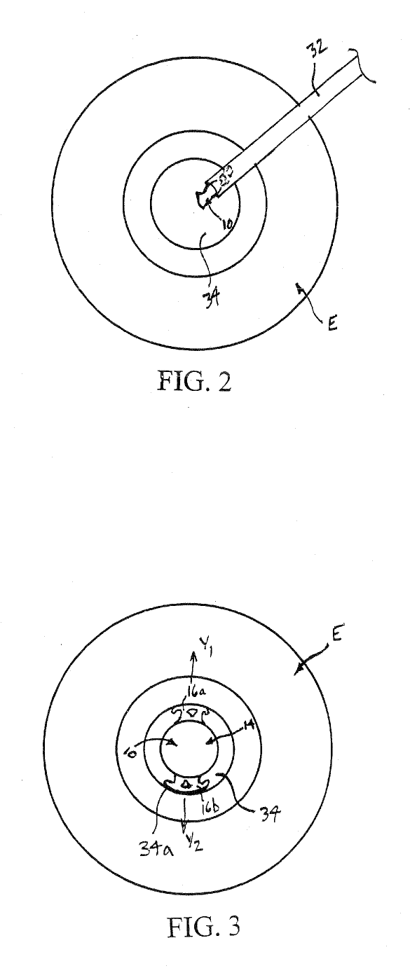

With reference to FIGS. 2 and 3, to install the IOL 10, a surgeon will make a small incision (˜2.5 mm in length) in the cornea or sclera the eye E. Next, he or she will fold or roll the IOL 10 and place it into a lens injector 32, as known in the art. The surgeon then inserts the injector 32 into the small incision until it reaches the capsular bag 34. At this point, the folded or rolled IOL 10 may be injected into the capsular bag 34. Over a short amount of time, the IOL 10 will naturally unfurl itself within the capsular bag 34. As it unfurls, the supports 16a, 16b of the IOL 10 engage the outer wall 34a of the capsular bag 34. As previously discussed, the supports 16a, 16b provide force in the direction Y₁ and Y₂, which positions the optic element 14 in the desired plane for focal correction. Additionally, these outward forces support the capsular bag 34, thereby assisting and minimizing capsular prolapse. Given the small incision, the natural internal pressure of the eye holds the incision closed, thereby alleviating the need for stitches. Although shown in FIG. 3 as substantially vertical, the IOL 10 may reside horizontally or at any angle/position in the capsular bag 34.

The foregoing descriptions of various embodiments of the invention are provided for purposes of illustration, and are not intended to be exhaustive or limiting. Modifications or variations are also possible in light of the above teachings. For example, the apertures 28a, 28b may be any shape to allow for the desired amount of outward force and/or bending. Alternatively, the supports 16a and 16b may be substantially solid and not include the apertures 28a and 28b. Additionally, the legs 20, 22 may be substantially straight and connected as a single piece of material. The curved members 26a, 26b may also be substantially straight. The IOL 10 may have any number of supports for positioning the optic element 14 in the desired position. The embodiments described above were chosen to provide the best application to thereby enable one of ordinary skill in the art to utilize the disclosed inventions in various embodiments and with various modifications as are suited to the particular use contemplated. All such modifications and variations are within the scope of the invention.

1. A lens for an eye, comprising:
an optic element; and
a plurality of symmetrical supports connected to the optic element, wherein the lens may be implanted in the eye.

2. The lens of claim 1, wherein the plurality of symmetrical supports are flexible.

3. The lens of claim 1, wherein the plurality of symmetrical supports comprise two supports positioned on opposite sides of the optic element.

4. The lens of claim 3, wherein the optic element is formed from first and second curved surfaces on opposite sides of a body.

5. The lens of claim 1, wherein the optic element comprises first and second curved surfaces.

6. The lens of claim 1, wherein the plurality of symmetrical supports each include a first leg and a second leg extending away from a body.

7. The lens of claim 6, wherein the first and second legs diverge from a center line of the body.

8. A lens for an eye, comprising:
a body having a curved optic element; and
a first and second support connected to the optic element, each support has a plurality of leg portions and the first support includes a first aperture, whereby the lens may be implanted in the eye.

9. The lens of claim 8, wherein the optic element comprises first and second curved surfaces formed on opposite sides of the body.

10. The lens of claim 8, wherein the optic element is positioned substantially in the center of the body.

11. The lens of claim 10, wherein the first and second supports are symmetrical.

12. The lens of claim 11, wherein the second support includes an aperture and the first and second supports may bend in a direction away from a plane defined by the body.

13. The lens of claim 8, wherein each of the leg portions terminates in a foot portion.

14. The lens of claim 13, wherein a first member connects the foot portions of the first support and a second member connects the foot portions of the second support.

15. The lens of claim 14, wherein the first and second members are curved and the legs of the first support and first member define the first aperture, and the legs of the second support and second member define a second aperture.

16. The lens of claim 15, wherein the apertures are substantially triangular.

17. A lens for an eye, comprising:
a body having a curved optic element; and
first and second symmetrical supports connected to the body, each support including an aperture and each support terminating in a curved member, wherein the lens may be implanted in the eye.

18. The lens of claim 17, wherein the body has both horizontal and vertical symmetry.

19. The lens of claim 17, wherein each support includes at least one foot portion that extends beyond a radius of the optic element.

20. The lens of claim 18, wherein each support includes an aperture.

21. A lens for an eye, comprising:
a body having first and second curved optic elements, the optic elements are positioned on opposite sides of the body;
first and second symmetrical supports connected to the body, each support including a plurality of legs, each of the legs curving away from a center line of the body;
a foot portion positioned at the end of each leg;
a first member residing between and connecting each of the foot portions of the first support;
a second member residing between and connecting each of the foot portions of the second support;
wherein each of the first and second symmetrical supports includes an aperture and the lens may be implanted in the eye.

22. The lens of claim 21, wherein each the foot portions extends beyond a radius of the optic elements.

23. The lens of claim 21, wherein the first and second symmetrical supports are flexible, the first and second members are curved, and the apertures are substantially triangular.

24. A method of implanting a lens inside an eye, comprising:
making an incision in a cornea or sclera of the eye;
inserting the lens into a capsular bag of the eye, wherein the lens includes an optic element and a plurality of symmetrical supports carried by the lens.

25. The method of claim 24, wherein the inserting step comprises rolling the lens and placing it into an injector.

26. The method of claim 24, wherein the inserting step comprises folding the lens and placing it into an injector.
